(19) United States
(12) Patent Application Publication
(10) Pub. No.: US 2006/0263296 A1
(43) Pub. Date: Nov. 23, 2006

(54) Diagnostic kit for the measurement of glucose metabolism, method of use, and method for the manufacture thereof

(76) Inventors: David Kinniburgh, St. Alberta (CA); John Porter, Edmonton (CA)

Correspondence Address: Isotechnika Inc., Suite 935, 1400 E. Southern Avenue, Tempe, AZ, 85282, US

(21) Appl. No.: 11/132,608

(22) Filed: May 18, 2005

(51) Int. Cl. A61K 49/00

(52) U.S. Cl. 424/9.1

(57) ABSTRACT

The present invention includes a kit for use in the performance of a ¹³CO₂ breath test of glucose metabolism which has quality controlled labeled sugar in a first container and unlabeled sugar in a second container, methods of use and methods of manufacture thereof.

### CROSS-REFERENCE TO RELATED APPLICATIONS

This application claims benefit of U.S. Application No. 60/572,189 filed May 17, 2004.

### FIELD OF THE INVENTION

The present invention relates to a diagnostic kit for the measurement of metabolism, including carbohydrate metabolic capacity and to a method for manufacturing and using such a kit. The kit provides a controlled amount of ¹³C-enriched glucose for use in a breath test for diagnosis of a metabolic disorder related to carbohydrate metabolism.

### REFERENCES

The following references are referred to by their numbers in parenthesis in this specification.
1. Porter et al. U.S. Pat. No. 5,912,178 issued Jun. 15, 1999
2. Kohno et al., U.S. Pat. No. 5,916,538 issued Jun. 29, 1999
3. Katzman, U.S. Pat. No. 5,962,335 issued Oct. 5, 1999
4. Ben-Oren et al., U.S. Pat. No. 6,656,127 issued Dec. 2, 2003
5. Becerro de Bengou Vallejo, U.S. Pat. No. 6,171,811 issued Jan. 9, 2001
6. Yatscoff et al., U.S. Pat. No. 6,461,870 issued Oct. 8, 2002
7. Yatscoff et al., U.S. Pat. No. 6,468,802 issued Oct. 22, 2002
8. Yatscoff et al., U.S. Pat. No. 6,599,750 issued Jul. 29, 2003
9. Yatscoff et al., U.S. Pat. No. 6,602,715 issued Aug. 5, 2003
10. EP0826377 March, 1998
11. EP0913161 May, 1999
12. Beck et al., Metabolic and Genetic Characterization of Pre-diabetic States. Sequence of Events Leading to Non-insulin-dependent Diabetes Mellitus. J Clin Invest, 94, (1994), pp. 1714-1721.
13. Bonora et al., Homeostasis Model Assessment Closely Mirrors the Glucose Clamp Technique in the Assessment of Insulin Sensitivity, Diabetes Care, 23, (2000), pp. 57-63.
14. CDC Diabetes Cost-Effectiveness Study Group, The Cost-Effectiveness of Screening for Type 2 Diabetes, JAMA, 280, (1998), pp. 1757-1763.
15. Emoto et al., Homeostasis Model Assessment as a Clinical Index of Insulin Resistance in Type 2 Diabetic Patients Treated With Sulfonylureas, Diabetes Care, 22, (1999), pp. 818-822.
16. The Expert Committee on the Diagnosis and Classification of Diabetes Mellitus, Report of the Expert Committee on the Diagnosis and Classification of Diabetes Mellitus, Diabetes Care, 24, (2001), suppl 1.
17. Gabir et al., The 1997 American Diabetes Assciation and 1999 World Health Organization Criteria for Hyperglycemia in the Diagnosis and Prediction of Diabetes, Diabetes Care, 23, (2000), pp. 1108-1112.
18. Ganda et al., Reproducibility and Comparative Analysis of Repeated Intravenous and Oral Glucose Tolerance Tests, Diabetes, 27, (1978), pp. 715-725.
19. Haffner et al., A Prospective Analysis of the HOMA Model: The Mexico City Diabetes Study, Diabetes Care, 19, (1996), pp. 1138-1141.
20. Harris, M. I., Undiagnosed NIDDM: Clinical and Public Health Issues, Diabetes Care, 16, (1993), pp. 642-652.
21. Helge et al., Carbon-13 Dioxide Breath Tests in Normal and Diabetic Children Following Ingestion of .sup.13 C-Glucose, Chemical Abstract Service, Database Accession No. 90:37276, XP002120262. (Abstract).
22. Hirai et al., The Breath Test Using Pure (1-.sup.13 C) Glucose: A New Simple Method for Evaluating the Glucose Oxidation of Capacity, Biosciences Information Services, retrieved from STN, XP002120264. (Abstract).
23. Hosker et al., Continuous Infusion of Glucose with Model Assessment: Measurement of Insulin Resistance and .beta.-cell Function in Man, Diabetologia, 28, (1985), pp. 401-411.
24. Ko et al., Use of the 1997 American Diabetes Association Diagnostic Criteria for Diabetes in a Hong Kong Chinese Population, Diabetes Care, 21, (1998), pp. 2094-2097.
25. Ko et al., The Reproducibility and Usefulness of the Oral Glucose Tolerance Test in Screening for Diabetes and Other Cardiovascular Risk Factors, Ann. Clin. Biochem., 35, (1998), pp. 62-67.
26. Lifschitz et al., Journal of Pediatric Gastroenterology and Nutrition, vol. 7(6), pp 842-847 (November-December 1988). (Abstract).
27. Lillioja et al., Insulin Resistance and Insulin Secretory Dysfunction as Precursors of Non-insulin-dependent Diabetes Mellitus; Prospective Studies of Pima Indians, New England Journal of Medicine, 329, (1993), pp. 1988-1992.
28. Martin et al., Role of Glucose and Insulin Resistance in Development of Type 2 Diabetes Mellitus: Results of a 25-year Follow-up Study, Lancet, 340, (1992), pp. 925-929.
29. Matthaei et al., Pathophysiology and Pharmacological Treatment of Insulin Resistance, Endocr. Rev., 21, (2000), pp. 585-618.
30. Matthews et al., Homeostasis Model Assessment: Insulin Resistance and Beta-cell Function from Fasting Plasma Glucose and Insulin Concentrations in Man, Diabetologia, 28, (1985), pp. 412-419.
31. McAuley et al., Diagnosing Insulin Resistance in the General Population, Diabetes Care, vol. 24, No. 3, (March 2001), pp. 460-464.
32. Meltzer et al., 1998 Clinical Practice Guidelines for the Management of Diabetes in Canada., Canadian Medical Association Journal, 159, (1998), (suppl. 8): S1-29.
33. Radziuk, J., Insulin Sensitivity and Its Measurement: Structural Commonalities Among the Methods, Journal of Clinical Endocrinol Metab., 85, (2000), pp. 4426-4433.
34. Riccardi et al., Reproducibility of the New Diagnostic Criteria for Impaired Glucose Tolerance, Am. J. Epidemiol, 121, (1985), pp. 422-429.
35. Rocker et al., Breath-by-Breath Measurements for the Analysis of Exogenous Glucose Oxidation During Intense Endurance Exercise (Using [¹³C]-Isotopes, Int. Journal of Sports Medicine, vol. 17, pp 480-486 (1996).
36. Tanis et al., Human Liver Glycogen Metabolism Assessed with a ¹³C-enriched Diet and a .sup.13 CO.sub.2 Breath Test, Biosciences Information Services, retrieved from STN, XP002120263. (Abstract).
37. World Health Organization, Prevention of Diabetes Mellitus: Report of a WHO Study Group, Geneva: WHO, (1994), Technical Report Series No. 844. World Health Organization, Definition, Diagnosis and Classification of Diabetes Mellitus and its Complications, Report of a WHO Consultation, Part 1, Diagnosis and Classification of Diabetes Mellitus, Geneva: WHO, (1999).
38. Lefebvre P et al., “Naturally Labeled 13C-glucose. Metabolic studies in human diabetes and obesity,” Diabetes 24(2):185-189 (1975).
39. Kato, et al. U.S. Pat. No. 5,352,590 issued Oct. 4, 1994
40. Isotope Tracers in Metabolic Research, Second Edition, by Robert R. Wolfe and David L. Chinkes, John Wiley & Sons, 2005, p. 2.
41. Yatscoff et al., U.S. Pat. No. 6,878,550 issued Apr. 12, 2005.

### BACKGROUND

The degree to which the body is able to utilize carbohydrates such as glucose to produce energy is referred to as carbohydrate metabolic capacity. This capacity is dependent on the activity of metabolic enzymes which break down energy sources, such as glucose, to form products, such as CO₂. Carbohydrate metabolic capacity is measurable by determining a patient's ability to utilize carbohydrates, such as glucose, as a source of metabolic energy.

An individual's carbohydrate metabolic capacity may be impaired due to genetic factors, environmental factors or due to matters of personal choice. Genetic disturbances in carbohydrate metabolic capacity usually result from the absence or impairment of a metabolic enzyme, resulting in the absence of the impaired enzyme's products, or the build-up of the enzyme's substrates. Examples of genetic metabolic disorders include von Gierke's disease, Pompe's disease, Forbes' disease, Andersen's disease, McArdle's disease, Hers' disease and Tarui's disease.

Disturbances in carbohydrate metabolism can also be acquired. Acquired carbohydrate metabolic disorders include diabetes. Type 1 diabetes is the result of the destruction of insulin-producing cells in the pancreas. Type 1 diabetes can result from viral infection, chemical exposure or autoimmune reactions. Additional causes are possible. Type 2 diabetes develops over time and results from a decreasing amount of insulin in the body, or decreasing responsiveness of the body to the insulin that is present. Diabetes can lead to life-threatening conditions from diabetic ketoacidosis to hyperosmolar coma and death. According to the American Diabetes Asosciation, 1.3 million new cases of diabetes are diagnosed annually among patients over the age of 20. The prevalence of diabetes in the general population is approximately 6-8%. Only about half of those having diabetes are actually diagnosed.

Diabetes is not a single disease, but an array of diseases that exhibit the common symptom of impaired glucose utilization. In general, the following types of diabetes have been recognized: type I diabetes mellitus, type II diabetes mellitus, secondary diabetes mellitus, impaired glucose tolerance and gestational diabetes mellitus. The general characteristics of the symptoms of diabetes include: polyuria (excretion of large quantities of urine); hyperglycemia (high blood glucose levels); glucosuria (abnormal presence of glucose in urine); polydipsia (excessive thirst); polyphagia (excessive hunger); and sudden weight loss.

Diabetes is a risk factor for a variety of conditions including heart disease, cerebrovascular stroke, neuropathy (nerve damage), nephropathy (kidney damage), retinopathy (eye damage), hyperlipidemia (excessive blood lipids), angiopathy (damage to blood vessels) and infection. It is recognized that typical diabetic complications are initiated in the “pre-diabetic” state (12, 20, 37). Early detection and treatment through lifestyle changes, diet modification or medication may help prevent or ameliorate such complications. Screening is recommended (16, 32, 37).

A reliable diagnostic test capable of distinguishing the degree to which a patient's carbohydrate metabolic capacity is compromised is needed. A number of different methods exist for determining a condition of intolerance for glucose. These include postprandial blood glucose, oral glucose tolerance test (OGTT), O'Sullivan glucose tolerance test (gestational test), hemoglobin Alc (HbA¹, HbA_{1c}), islet cell antibodies, glutamic acid decarboxylase GAD) antibodies and insulin antibodies. Diabetes is most readily detected when the carbohydrate metabolic capacity is tested. This is done by stressing the system with a defined glucose load as in the oral glucose tolerance test (OGTT). The OGTT has been criticized, however, because many of the variables affecting test results are difficult to control. For instance: patients must be on a standardized carbohydrate diet at least three days before the test; the test requires an 8 to 16 hour fast; the test should only be performed on ambulatory patients; stress should be avoided; exercise should be avoided; various hormone imbalances can affect validity such as with: thyroxine, growth hormone, cortisol and catecholamines; various drugs and medications can affect validity such as: oral contraceptives, salicylates, nicotinic acid, diuretics and hypoglycemics; and evaluation should normally be corrected for age. The greatest disadvantage of the OGTT is that it is poorly reproducible and this limits its diagnostic usefulness.

Breath tests for measuring glucose metabolism are non-invasive and have been shown to be useful diagnostically. Rocker et al., (35) disclose the use of a breath test for the analysis of the onset of exogenous glucose oxidation during exercise using naturally labeled ¹³C-glucose. Lefebvre P, et al (38) disclose the use of naturally labeled ¹³C-glucose for metabolic studies in human diabetes and obesity. Katzman et al. (3,4) disclose the use of galactose, xylose, fructose or lactic acid labeled at one position as a breath test for diagnosing diabetes. Kohno et al., (2) disclose administration of glucose labeled with ¹³C in one specific position for diagnosing diabetes.

Yatscoff et al. (6, 7, 8, 9, 41) disclose a ¹³C-glucose breath test and kit for measuring glucose metabolism, the oxidation of glucose and subsequent emission of ¹³CO₂ However, there exists a need to provide a ¹³CO₂ breath test kit which contains a controlled, reproducible, predictable and standard amount of ¹³C enriched glucose, a method of making and using same.

### SUMMARY OF THE INVENTION

An embodiment of the present invention is a kit for use in the performance of a ¹³CO₂ breath test of glucose metabolism which has quality controlled labeled sugar in a first container and unlabeled sugar in a second container. In embodiments, the container may be a cup, a vial, a bottle, or a flask which may have a fill line for indicating an amount of liquid to be added.

In another embodiment, the kit of the present invention may contain uniformly labeled ¹³C-glucose, ¹³C-glucose which is labeled at least at one position, or ¹³C-glucose which is randomly labeled.

An embodiment of the kit of the present invention may also contain a breath collection device. The breath collection device may be a breath container or a straw.

In yet another embodiment of the kit of the present invention, the quality controlled labeled sugar may be mixed with an inert material. In further embodiments, the inert material may be lactose, mannitol, talc, magnesium stearate, sodium chloride, potassium chloride, citric acid, spray-dried lactose, hydrolysed starches, starch, microcrystalline cellulose, cellulosics, icodextrin, calcium sulphate, and dibasic calcium phosphate, and these materials may be mixed with other compounds such as flavoring agents, preservatives, and the like. An embodiment of the present invention may also contain instructions.

An embodiment of a method for manufacturing the kit of the present invention includes the steps of:
a. weighing an amount of labeled sugar;
b. weighing an amount of filler material;
c. mixing the labeled sugar with the filler material;
d. providing a first quality control measurement to ensure that the labeled sugar and the filler have been uniformly mixed;
e. dispensing a weighed amount of uniformly mixed labeled sugar and filler into a container;
f. repeating steps a through e to provide a plurality of containers each containing the uniformly mixed labeled sugar and fillers; and,
g. providing a second quality control measurement to ensure that the containers contain an expected weighed amount of uniformly mixed labeled sugar and filler;

In further embodiments of the method of manufacturing the kit of the present invention, the method may also include a step of sealing the container. In additional embodiments, the method of manufacturing the kit of the present invention includes quality control steps. In further embodiments, the quality control measurements are performed by enzymatic assay.

An embodiment of a method of using the kit of the present invention includes the steps of:
1) obtaining a baseline breath sample from a subject;
2) calculating a first ¹³C to ¹²C ratio value from the baseline breath sample from the subject
3) mixing the quality controlled labeled sugar with the unlabeled sugar and a liquid to form a solution;
4) administering the solution to the subject;
5) obtaining a second breath sample from the subject;
6) calculating a second ¹³C to ¹²C ratio value from the second breath sample;
7) calculating a delta value by subtracting the first ¹³C to ¹²C from the second ¹³C to ¹²C; and
8) comparing said delta value to standardized delta values to obtain a diagnosis.

### BRIEF DESCRIPTION OF THE DRAWINGS

FIG. 1 provides a flow chart of a method for manufacturing an embodiment of the kit of the present invention.

FIG. 2 provides a flow chart of a method of using an embodiment of the kit of the present invention.

FIG. 3 illustrates an embodiment of the diagnostic kit of the present invention.

### DETAILED DESCRIPTION OF THE INVENTION

Embodiments of the present invention provide a kit for determining carbohydrate metabolic capacity or glucose metabolism by measuring exhaled carbon dioxide derived from the metabolism of glucose (referred to as a ¹³CO₂ breath test), a method for manufacturing the kit, and a method for using the kit. Carbohydrate metabolic capacity or glucose metabolism refers to the ability of a subject to utilize a carbohydrate; to turn a sugar into metabolic energy, CO₂, and water. A subject with a normal carbohydrate metabolic capacity is very efficient at turning sugar into these metabolic by-products. Therefore, when a normal subject is provided with a labeled sugar, the labeled sugar is efficiently turned into labeled by-products. When a normal subject is provided with sugar labeled with ¹³C, the labeled sugar is efficiently turned into ¹³CO₂. A subject with an impaired ability to turn sugar into its metabolic by-products may have a low metabolic rate, may be in a pre-diabetic condition, or may have diabetes. A diagnostic test, such as a ¹³CO₂ breath test may provide extremely important early information about a subject's metabolic condition that may allow the patient to make changes in diet and exercise or seek medical or pharmaceutical help, which may lead the patient back to a normal metabolic capacity.

The exact location of a tracer molecule within a molecule is denoted by the numbering of the molecule. Isotope tracers or isotopically labeled atoms are referred to by identification of the weight of the atom by a superscript prior to the letter. ¹²C, ¹³C and ¹⁴C refer to carbon atoms with atomic masses of 12, 13, and 14, respectively. ¹²C is the most abundant carbon atom, approximately 99%. ¹³C is a naturally occurring stable isotope. ¹⁴C is a radioactive isotope. The position within the molecules of an enriched carbon tracer is denoted by the appropriate carbon number and the description of the isotope used as a tracer. 1-¹³C₁-glucose refers to a molecule of glucose which has a single ¹³C lable at the 1 position of the glucose. The subscript following the C refers to the number of specifically enriched atoms of carbon in the molecule. If the 1 and 2 positions were both specifically labeled, the compound would be 1,2-¹³C₂-glucose. If all carbons are labeled, it is considered to be uniformly labeled, which is abbreviated by U. Thus a glucose molecule with all carbon atoms labeled is denoted as U-¹³C₆-glucose, U-¹³C-glucose or uniformly labeled ¹³C glucose (40) Glucose is metabolized by many different enzymes, releasing ¹³C to form ¹³CO₂ from all positions of the glucose molecule. Providing a subject with uniformly labeled glucose maximizes the signal that will be produced in a breath test which measures ¹³CO₂.

Kohno et al. (2) and Kato et al. (39) describe ¹³C labeled glucose which has been labeled at least at one specific position, for example glucose that has ¹³C in position 1 on the glucose molecule. Yatscoff et al. (6, 7, 8, 9, 41) teach a ¹³CO₂ breath test kit for measuring carbohydrate metabolic capacity or glucose metabolism, that includes a predetermined quantity of ¹³C enriched-glucose mixed in a container with a larger quantity of unlabeled glucose. When it was time to administer the test, the contents of the container were mixed with water to form a solution, and the subject drank the solution. In an example, the breath test was performed by obtaining a baseline breath sample from a subject, administering a drink containing 25 mg of ¹³C enriched glucose and 15 grams of unlabeled glucose to the subject, waiting 1.5 hours and then taking a second breath sample. The ratio of ¹³CO₂/¹²CO₂ was measured before and after the administration of ¹³C labeled glucose. If the patient's glucose metabolism or carbohydrate metabolic capacity was normal, an increase in the ratio of ¹³CO₂ to ¹²CO₂ was expected after ingesting ¹³C labeled glucose. If the patient had impaired glucose metabolism or carbohydrate metabolic capacity, the ratio of ¹³CO₂ to ¹²CO₂ was decreased with respect to normal. That is, the patient was not as efficient at turning labeled carbohydrate into labeled by-products.

According to Yatscoff et al. (6, 7, 8, 9, 41), to form the sugar solution that was administered to patients in the ¹³CO₂ breath test kit, 25 mg of uniformly labeled ¹³C glucose was weighed out and placed in a container having a liquid fill line. 15 g of unlabeled glucose was weighed out and added to the same container. Then, before the test was administered, water was added to form a drink which was administered to the subject.

While this method for producing an accurate solution from a mixture of ¹³C labeled glucose and unlabeled sugar was adequate for the production of single kits or small batches of kits, when attempts were made to increase the production size of the kits, or mass produce the kits, this method of production proved inefficient. In addition, it became clear that this method of producing the source of ¹³C labeled glucose was not amenable to quality control measures and therefore would not produce reproducibly accurate and consistent kits, and would not be approved by regulatory authorities. For example, once small amounts of labeled glucose (25 mg) are mixed with relatively large amounts of unlabeled sugar (15 g), it is very difficult to test the mixture to determine if the proper amount of labeled sugar has been provided to the mixture. Because the amount of labeled sugar is so small compared to the amount of unlabeled sugar, it is difficult to test this sugar mixture for the amount of “label” present in the mixture. It is also extremely difficult to separate sugar labeled with ¹³C from unlabeled sugar once they are mixed together. Quality control measures designed to ensure that the proper amount of label is administered in each kit is very difficult for kits manufactured in this way.

In addition, because the kits described in Yatscoff (ibid) measure small changes in the ¹³CO₂/¹²CO₂ ratio, the administration of precisely controlled amounts of ¹³C labeled glucose is very important in ensuring the accuracy and effectiveness of the test. And, the amount of ¹³C labeled glucose to be administered per kit is a very small amount, approximately 25 mg. It is difficult to accurately and reproducibly measure such a small amount of dry sugar for bulk production of these kits. A quality control process to ensure that the precise amount of labeled glucose was provided to each kit, for the bulk production of breath test kits to measure carbohydrate metabolic capacity or glucose metabolism was needed. And, a kit that provides an accurate and reproducible amount of labeled sugar and is amenable to quality control measures was also needed.

One way to provide labeled sugar to a ¹³CO₂ breath test kit in a way that is amenable to quality control measures is to separate the labeled sugar from unlabeled sugar in the kit. Labeled sugar, provided in a separate container, is amenable to quality control measures. For example, as the kits are assembled, a specific amount of labeled sugar, an aliquot, can be delivered to a container. The container may be a cup, a vial, a bottle, or a flask. Many aliquots of labeled sugar can be delivered to many containers. The containers can be placed into kits. Quality control measures can be performed on labeled sugar provided in this way. Quality control measures can involve, for example, removing a random container from a batch of containers containing labeled sugar and measuring the amount of labeled sugar in the randomly selected container to be sure that the appropriate amount of labeled sugar is in the container. The amount of labeled sugar present can be measured by measuring the amount of ¹³C present by, for example, weighing the amount of labeled sugar provided. These quality control measures can be performed before or after the labeled sugar is delivered to the kit. For example, a random container containing an aliquot of labeled sugar can be removed from a batch of such containers as they are assembled. Or, the containers can be delivered to the kits and the kits can be assembled to include a separate container containing unlabeled sugar. As a quality control measure, a kit can be removed from a batch of kits, the container containing the labeled sugar can be removed from the kit, and quality control measures can be taken to ensure that the container contains the appropriate amount of labeled sugar. Regulatory bodies and medical professionals administering ¹³CO₂ breath tests can be assured that the test results obtained using kits assembled in this manner, including quality control steps, will be based on the administration of a specific amount of labeled sugar.

Labeled sugar that has been measured and delivered to containers, and that has undergone quality control measures as described above and below, to ensure that the appropriate amount of labeled sugar has been delivered to the containers, is quality controlled labeled sugar.

Labeled sugar can be provided in the kit in dry form or in liquid form. For example, labeled sugar can be dissolved in liquid, water for example, at a specific concentration. An aliquot of liquid labeled sugar can then be provided in the kit in a container. In the same way, a container of liquid labeled sugar can be randomly removed from a production batch, either before or after the container is packaged with the kit, and the amount of labeled sugar present in the liquid aliquot of labeled sugar solution can be measured to ensure that the appropriate amount of labeled sugar has been delivered to that batch of containers or kits. Liquid labeled sugar can be measured for the amount of ¹³C present by, for example, spectrophotometry. Kits that have gone through this quality control process to ensure that the amount of labeled sugar present in the kit is accurate, are quality controlled kits.

Because the amount of labeled sugar provided in the ¹³CO₂ breath test kit of the present invention is very small, for example 25 mg, it can be difficult to accurately measure. In a bulk assembly environment, accurate measurement of these small amounts of dry ingredients can be difficult. One way to reduce the difficulty of measuring a small amount of dry ingredient is to combine the dry ingredient with filler to “bulk up” the dry ingredient. When a small amount of target material, in this case labeled sugar, is combined with a filler ingredient, the actual amount of material to be measured is greater, thereby reducing errors and risks associated with the measurement of a very small amount of material. Mixing the labeled sugar with a filler material, therefore, may make the accurate and reproducible measurement of a small amount of a dry ingredient less difficult.

Filler may be any material that is inert for the purposes of the ¹³CO₂ breath test kit of the present invention. Inert materials suitable for mixing with the ¹³C-glucose include binders, fillers, flavoring agents, thickening agents, coloring agents, emulsifiers, and the like with the proviso that the material does not test positive for glucose in a glucose assay. Fillers useful for mixing with the ¹³C-glucose include but should not be limited to lactose, mannitol, talc, magnesium stearate, sodium chloride, potassium chloride, citric acid, spray-dried lactose, hydrolysed starches, starch, microcrystalline cellulose, cellulosics, icodextrin, calcium sulphate, dibasic calcium phosphate and mixtures thereof.

Other materials may also be added to the labeled sugar provided in the ¹³CO₂ breath test kit of the present invention. For example, preservatives, coloring agents and/or flavoring agents may be added.

In addition to the labeled sugar, an amount of unlabeled sugar (15 g) can be provided in the ¹³CO₂ breath test kit to provide a “glucose challenge.” However, in order to provide the opportunity for quality controlling the amount of labeled sugar provided in the kit, the labeled sugar can be provided in a first container, unlabeled sugar can be provided in a second container, and both containers can be provided in a kit, for administration to a subject.

The container which provides the labeled carbohydrate may be sealed. For example, the container may have a screw top, a pop-top, a tab top, a plastic seal, or any other mechanism for sealing the container to prevent the labeled sugar from spilling from the container during or after the process of assembling the kit. Unlabeled sugar can be weighed and provided in a breath test kit in a container, to provide an exact amount of unlabeled sugar in the kit.

In order to manufacture ¹³CO₂ breath test kits which are amenable to quality control measures, the labeled sugar is measured and provided in a container. However, in order to accommodate the quality control steps, the process of providing the labeled sugar in a container may include additional steps.

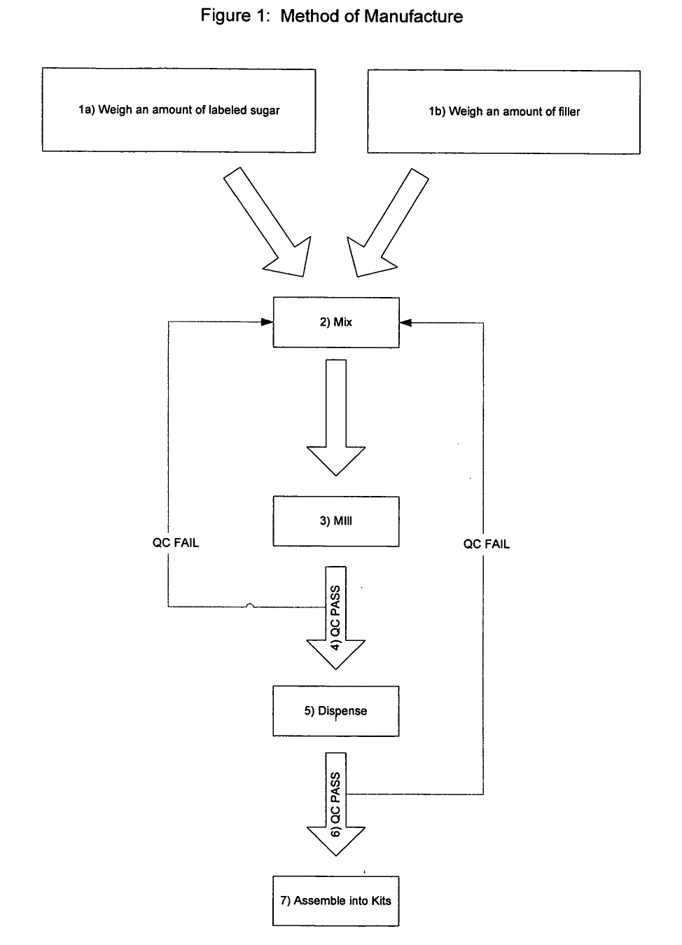

FIG. 1 illustrates an embodiment of a method of manufacturing the ¹³C breath test kit of the present invention. Labeled sugar is mixed with filler such as mannitol. Mannitol is an acceptable filler or excipient because mannitol is not metabolized in the body like sugar, and because it does not react in tests or processes that measure the amount of sugar, including labeled sugar, present in a sample. The ingredients are mixed together, milled to a uniform particle size, mixed again, quality tested to be sure that mixture is uniform, dispensed into container, and the containers are again quality tested to be sure that each container in the batch contains the proper amount of labeled sugar.

FIG. 1 illustrates a method of manufacturing a kit containing quality controlled amounts of labeled sugar. FIG. 1 illustrates that an appropriate amount of sugar in step 1a) and filler in step 1b) are measured and added to a hopper to be mixed in step 2. Optionally, other materials such as flavoring, preservatives, or other materials can also be added prior to the mixing step. In step 3, these materials are then milled in, for example, a Fitz Mill, (Model DAS06 ser. # 11273; Fitzpatrick Co., Elmhurst, Ill.) to a desired particle size to ensure that the mixture is of uniform particle size. Uniform particle size is important to ensure that when the material is dispensed, the dispensing step is not adversely affected by non-uniform particles in the mix. For example, larger, heavier particles may fall to the bottom of the hopper, so that when the material is dispensed, heavier, larger particles may be dispensed first and lighter, smaller particles dispensed last, resulting in a batch of dispensed aliquots that are not uniform. Alternatively, the milling step may be performed before the mixing step. Or, the materials may be mixed, then milled, then mixed again. Those of ordinary skill in the art will recognize that there are many variations in the order of these steps. For example, the step of milling may not be necessary, if quality control measures indicate that the appropriate amount of the labeled sugar or labeled sugar mixture is being dispensed in the absence of a milling step.

Once the ingredients are milled and mixed, a quality control test is performed in step 4 to ensure that the mixture is uniform. For example, a sample can be taken from the top, middle and bottom of the hopper, and tested for the presence of labeled sugar, in the appropriate amount. This is an in-process quality control step or a mixture quality control step. The step of quality control or testing that the proper amount of sugar is present in the sugar mixture, may be performed using any of several methods known in the art. Testing may be by weighing, by enzymatic methods, by radiometric method and/or by use of a sensor such as a biosensor. Enzymatic methods include the oxidase/peroxidase test and the hexaokinase/G6PDH test. Kits based upon oxidase/peroxidase or on hexokinase/G6PDH enzymatic procedures are available from Megazyme International Ireland Ltd., Wicklow, Ireland, Molecular Probes, Eugene, Oreg. (Amplex® Red Glucose/Glucose Oxidase Kit), and Sigma Chemical Co. Glucose sensors are available from such companies as NEC Corp.

When the mixture is tested prior to dispensing it into a receptacle and the test results indicate that a uniform mixture is not present (QC FAIL), then the process of mixing the ¹³C-glucose mixture and sampling may be repeated, or the mixing step may be extended so that more mixing takes place. For example, the labeled sugar mixture can be mixed for one hour and tested to ensure that the mixture is uniform. If the mixture is not uniform, and the mixture fails to fall within the specifications of the quality control parameters, the mixing step can be extended for an additional period of time. For example, if the mixture is not uniform, it can be mixed for an additional hour. So, as a result of mixing and testing for uniform mixtures, a time window for the mixing step can be established.

If the mixture passes the quality control measurements, the process can proceed to the next step, Step 5, dispensing the mixture into containers. The mixed, milled and quality controlled mixture is dispensed into containers, in equal aliquots, one aliquot per container until all of the mixture has been dispensed, and a batch of containers is provided. An additional quality control measure is taken at step 6. A random sampling of containers can be taken for testing throughout the filling or dispensing process. For example, a container can be removed at the beginning, middle and end of a dispensing run, to ensure that each of the randomly selected containers, and therefore each of the containers in the batch, contains the appropriate amount of labeled sugar in a labeled sugar mixture. This is an additional quality control step, a batch quality control step or a dispensing process quality control step.

If the batch of containers containing an aliquot of labeled sugar passes this quality control measure, the batch is approved to be assembled into ¹³CO₂ breath test kits. The containers are sealed to prevent spilling the quality controlled material. This sealing step can occur before or after the batch quality control measures. If the batch of containers does not fall within the quality control specifications at step 6 (QC FAIL), the batch can either be discarded or the batch ca be replaced into the hopper, and re-mixed to enter the process again.

Labeled sugar that has been mixed with fillers or other materials and has undergone any of the quality control measures discussed above, is quality controlled labeled sugar mixture.

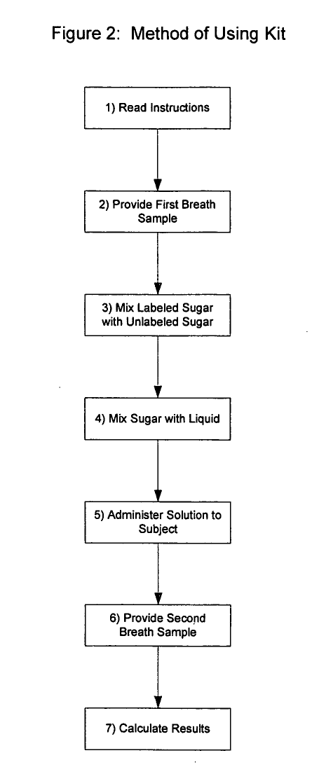

FIG. 2 provides a flow chart of a method of using an embodiment of the kit of the present invention. As a first step, the administrator of the test reads the instructions. Of course, an experienced administrator may not need to read the instructions provided with the kit, and the kit of the present invention may or may not contain instructions. If the step of reading the instructions is not necessary, or if instructions are not provided with the kit, step 1 may be skipped.

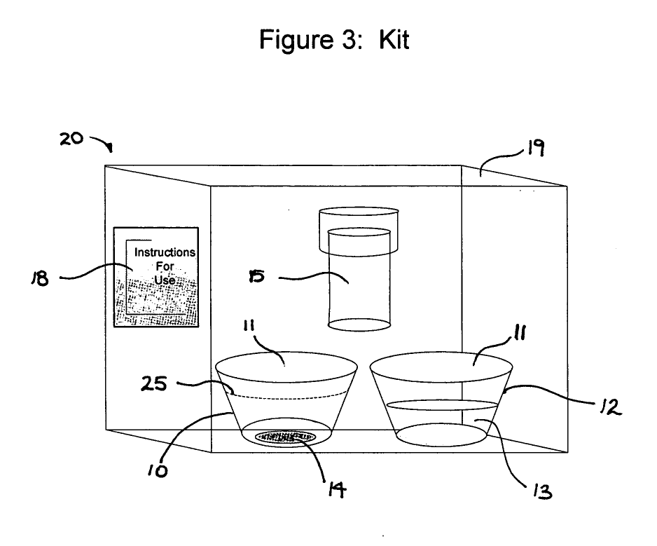

The instructions may include directions, for example, that the patient is to fast for at least 8 hours prior to providing a first baseline breath sample in a first breath sample container using a straw to direct the breath into the container. Further, the instructions may state that the dextrose from a second cup (12, see FIG. 3) is to be poured into a first cup (10, see FIG. 3), mixed with labeled sugar, and water added to a fill line (25, see FIG. 3) to make a drink. The instructions may instruct that the drink is to be consumed by the subject after the subject has provided the baseline breath sample. The instructions may also instruct that after a period of time, for example 1.5 hours after consumption of the solution, a second breath container and straw are to be used to obtain a second breath sample from the subject who has consumed the liquid. The instructions may further state that during the 1.5 hours, the patient only is permitted to drink small amounts of water. The instructions may also instruct that the two breath samples are to be mailed to the lab designated on the mailer for testing.

In step 2 of FIG. 2, the subject provides a first breath sample, or control breath sample. This breath sample may be provided using a breath collection device provided in the kit, or may be provided using a breath collection device that is not provided in the kit. The breath collection device may be, for example, a vial (see 15 FIG. 3). The breath collection container may also be a straw or a tube to direct breath directly into a ¹³CO₂ measuring device, a straw or a tube to direct breath into a breath container for later administration to a ¹³CO₂ measuring device, or a breath container or bag for later administration to a ¹³CO₂ measuring device. In step 3, labeled sugar is mixed with unlabeled sugar. The labeled sugar may be provided by itself or mixed with filler, flavoring agent, preservatives, or other ingredients. Either the labeled sugar or unlabeled sugar can be delivered in the kit in a container with a water fill line so that the proper dilution of sugar in water is marked on the container and the sugar is easily mixed with the appropriate amount of water. In step 4, the sugar is mixed with liquid, such as water. The subject is then administered 13C-labeled and unlabeled sugar in step 5. After an appropriate period of time, the subject provides a second breath sample in step 6. Both breath samples are analyzed, and the results are calculated in step 7. A delta value is calculated as the difference in the ¹³CO₂/¹²CO₂ ratio before and after the administration of the labeled sugar. By comparing the calculated delta value to population information, a diagnosis can be achieved. For example, if the calculated delta value correlates to the calculated delta values for patients with known carbohydrate metabolic conditions such as insulin resistance, type I or type II diabetes, a diagnosis of these conditions can be reached.

FIG. 3 illustrates an embodiment of the diagnostic kit 20 of the present invention. FIG. 3 illustrates that an embodiment of the kit 20 of the present invention may include a first container 10 which contains labeled sugar 14. Labeled sugar 14 may be provided in a mixture with fillers, flavoring agents, preservatives, or other ingredients, or labeled sugar may be provided alone in the first container 10. The first container 10 may have a cap or seal 11, to prevent the contents from spilling. First container 10 may also have a fill line 25 to indicate the amount of liquid, water, for example, to add to the first container to provide the appropriate amount of liquid sugar solution to administer to a subject. Second container 12 may also have a fill line (not shown).

FIG. 3 also illustrates a second container 12 which contains an amount of unlabeled sugar 13. The second container 12 may be identical to the container containing labeled sugar, 10, or it may be different. For example, if the unlabeled sugar 13 is provided in liquid form, container 12 may be a bottle. Container 12, containing unlabeled sugar 13 may also have a lid, 11, to prevent the contents from spilling.

Also present in this embodiment of the kit 20 of the present invention, is a breath collection device 15. In this embodiment the breath collection device 15 is a vial. As discussed above, the breath collection device may be a tube, a straw, a bag, or any device structured and arranged to assist the subject in providing a breath sample to a measuring device such as a spectrophotometer. Or alternatively, the breath collection device is a container for holding a breath sample for transport to a spectrophotometer. The breath collection device may be a bag that can be sealed and sent to a remote lab for spectrophotometric analysis of the breath sample to measure the concentration of ¹³CO₂. In addition, FIG. 3 illustrates that an embodiment of the kit 20 of the present invention may also include instructions 18, shown here on the outside of box 19, containing the contents of the kit.

The disclosures in this application of all articles and references, including patents, are incorporated herein by reference. The invention is illustrated further by the following example which is not to be construed as limiting the invention in scope or spirit.

### EXAMPLE

### Two Cup Breath Test Kit

To obtain sufficient uniformly labeled ¹³C-glucose mixture for approximately 2500 kits, 62.5 grams of ¹³C-glucose (uniformly labeled by substitution of each ¹²C with a ¹³C; GMP, ISOTEC, 3858, Miamisburg, Ohio), 62.5 grams of orange flavor crystals (Embassy Food Specialties LTD, Etobicoke, Ontario, Canada), and 14.875 kg of mannitol 60, (Pearlite® 160C, Multichem Inc., Boucherville, Quebec, Canada) were thoroughly mixed in the hopper of a figure eight blender (Model 10, Shatz 88 Inversion Mixer, serial # 98077; Inversion Machines; Ponoka, Alberta T5J1R2), milled to obtain a uniform particle size of less than about 0.024 inch diameter (Fitz Mill, (Model DAS06 ser. # 11273; Fitzpatrick Co., Elmhurst, Ill.), and then mixed again. A series of samples of the mixture were obtained from the top, middle, and bottom of the mixer and analyzed enzymatically using hexokinase (Sigma Chemical, St. Louis, Mo.) to determine if the labeled glucose was uniformly mixed, as a quality control measure. When the samples taken from the top portion of the mixture did not fall within the specified acceptable range of from 22.5-27.5 mg¹³C glucose/6 g powder, (actual measurement was 21.62 mg ¹³C glucose/6 g powder), after mixing for a total of two hours, the mixing time was increased to a total of three hours. When mixed for three hours, all measurements were within specifications. After determining that the labeled sugar was uniformly mixed, the batch was transferred to the hopper of a dispenser (Mateer Burt Auger Filler, Model 1800D; GEI Mateer Co.; Wayne, Pa.); aliquots of 6 grams each were placed into individual GMP polypropylene vials (B 1202-VOStarplex Scientific Inc.; Etobieoke, Ontario); capped with a screw on cap (B1202-1W—CO; Starplex Scientific Inc.; Etobieoke, Ontario); and sealed with tamper-proof sealing. Random vials containing labeled sugar were removed from the dispenser and weighed to ensure that the appropriate amount of labeled sugar was present in each container.

Dextrose as unlabeled sugar (USP Grade; Calcdon; Georgetown, Ontario) was provided to the kit in a second container (Starplex Scientific Inc.). The dextrose was weighed, milled, and dispensed in aliquots of 15 grams per cup. Each cup of isolated dextrose was provided with a peel-away lid.

Two distinguishable breath sample containers, two straws, a cup containing dextrose (unlabeled sugar) and a vial of quality controlled uniformly labeled ¹³C-enriched glucose-mannitol mixture were packaged together with instructions to form a quality controlled kit.

What is claimed is:

1. A diagnostic kit for use in the performance of a ¹³CO₂ breath test of glucose metabolism comprising:
quality controlled labeled sugar in a first container and unlabeled sugar in a second container.

2. The kit of claim 1 wherein the first container is selected from the group consisting of a cup, a vial, a bottle, and a flask.

3. The kit of claim 2 wherein the first container has at least one fill line for indicating an amount of liquid to be added.

4. The kit of claim 1 wherein the quality controlled labeled sugar is ¹³C-glucose which is uniformly labeled.

5. The kit of claim 1 wherein the quality controlled labeled sugar is ¹³C-glucose which is labeled at least at one position.

6. The kit of claim 1 wherein the quality controlled labeled sugar is ¹³C-glucose which is randomly labeled.

7. The kit of claim 1 wherein the unlabeled sugar is present in an amount sufficient to affect a glucose challenge.

8. The kit of claim 1 further comprising a breath collection device.

9. The kit of claim 8 wherein the breath collection device is a breath container.

10. The kit of claim 8 wherein the breath collection device is a chamber of a spectrophotometer.

11. The kit of claim 1 wherein the quality controlled labeled sugar is mixed with an inert material.

12. The kit of claim 111 wherein the inert material is selected from the group consisting of lactose, mannitol, talc, magnesium stearate, sodium chloride, potassium chloride, citric acid, spray-dried lactose, hydrolysed starches, starch, microcrystalline cellulose, cellulosics, icodextrin, calcium sulphate, and dibasic calcium phosphate.

13. The kit of claim 11 wherein the inert material comprises mannitol.

14. The kit of claim 13 wherein the inert material further comprises a flavoring agent.

15. The kit of claim 1 wherein the unlabeled sugar is provided in liquid solution.

16. A method for manufacturing a kit for use in the performance of a ¹³CO₂ breath test of glucose metabolism, said method comprising the steps of:
a. weighing an amount of labeled sugar;
b. weighing an amount of filler material;
c. mixing the labeled sugar with the filler material;
d. providing a first quality control measurement to ensure that the labeled sugar and the filler have been uniformly mixed;
e. dispensing a weighed amount of uniformly mixed labeled sugar and filler into a container;
f. repeating steps a through e to provide a plurality of containers each containing the uniformly mixed labeled sugar and fillers; and,
g. providing a second quality control measurement to ensure that the containers contain an expected weighed amount of uniformly mixed labeled sugar and filler.

17. The method of claim 16 wherein said first quality control measurement is performed by enzymatic assay.

18. The method of claim 16 wherein said second quality control measurement is performed by enzymatic assay.

19. A method of testing a subject using the diagnostic kit of claim 1 comprising the steps of:
a. obtaining a baseline breath sample from a subject;
b. calculating a first ¹³C to ¹²C ratio value from the baseline breath sample from the subject;
c. mixing quality controlled labeled sugar with unlabeled sugar and a liquid to form a solution;
d. administering the solution to the subject;
e. obtaining a second breath sample from the subject;
f. calculating a second ¹³C to ¹²C ratio value from the second breath sample;
g. calculating a delta value by subtracting the first ¹³C to ¹²C ratio from the second ¹³C to ¹²C ratio;
h. comparing said delta value to standardized delta values to obtain a diagnosis.
